(19) United States
(12) Patent Application Publication
(10) Pub. No.: US 2026/0171200 A1
(43) Pub. Date: Jun. 18, 2026

(54) PERMISSION-BASED SYSTEM FOR AND METHOD OF MANAGING ELECTRONIC MEDICAL RECORDS IN A COMPUTING ENVIRONMENT

(71) Applicant: Justin Lawrence SY, Tondo Manila (PH)

(72) Inventor: Justin Lawrence SY, Tondo Manila (PH)

(21) Appl. No.: 18/710,208

(22) PCT Filed: Nov. 14, 2022
(86) PCT No.: PCT/PH2022/050020

§ 371 Date: May 15, 2024

(30) Foreign Application Priority Data
Nov. 15, 2021 (PH) 12021050574
Nov. 15, 2021 (PH) 22021051326

(51) Int. Cl. G16H 10/60

(52) U.S. Cl. CPC G16H 10/60

(57) ABSTRACT

Disclosed are system for and method of managing electronic medical records in a computing environment, which are computer-based, and may be utilized as permission-based implementations for managing electronic medical records utilizing a set of computer-executable software application services in an electronic computing environment. The system, in one aspect, is adapted for keeping, sharing, enumeration, and development of the electronic medical records. The system utilizes a first permission level associated with a patient computing device and a second permission level associated with a medical data generation source. The first and second permission levels may be defined based on one or more privacy preference data and one or more access preference data from the patient computing device. The privacy and access preference data are indicative, respectively, of the privacy and access preferences of a patient associated with the patient computing device. The patient associated with the patient computing device is assigned a unique code so as to hide the identity of the patient, and the unique code is replaceable.

### TECHNICAL FIELD

The present invention relates to system for and method of managing electronic medical records in a computing environment and more particularly to such aspects that are permission-based and can be utilized for at least keeping, sharing, enumeration, and development of on-demand electronic mediscal records.

### BACKGROUND ART

There are generally two ways that a patient's medical records are digitally handled, either the data is in the possession of the patient, or it isn't. For systems where the data is with or controlled by the patient or Data-On-Patient (DOP), ease of access and versatility becomes an issue. These DOP systems mostly do not allow other parties to gain access to the data and are simply a personal records system where the patients themselves, who may not be the best qualified, may not encode medical data into their medical records in a clear and optimal manner.

DOP systems, or any comparatively similar systems, with added features to allow third-party access, require the patient to grant permission before providing viewing access and/or requires designated computers or equipment. These introduce barriers of use such as introducing too many additional steps and effort on the part of the patient and medical data generation sources (MDGS) to adopt these types of systems which is detrimental in the case of an emergency. These steps hinder the adoption of new systems that could be beneficial for the medical service provider and/or one or more of its associates and the patient.

Such as in the case of related system, method, and article of manufactures disclosed in U.S. Pat. No. 10,841,286 issued on 17 Nov. 2021 to Ilya Aronovich, where the system requires the patient to allow access to their records every time a new MDGS needs to. This system also adds steps by keeping track of the devices that the medical service provider uses as part of the authentication process, which the medical service provider might not desire for privacy reasons. The issue with this type of DOP system is that patients who are incapacitated will not be able to provide access and/or would not allow the most qualified persons (their doctors, medical providers, etc.) to encode their medical data for them. In most cases, the medical specialists are best equipped to encode the patient's data in a manner that is clear and organized to other medical specialists.

On the other hand, the data is not with the patient but with the healthcare system or Data-On-System (DOS) where on-demand-availability, privacy, and data verification becomes an issue. Patients can have multiple doctors or medical care providers who use different systems which makes compiling and sorting medical data very difficult especially in time-sensitive emergency situations. Since the medical data are also spread out to different parties and are attached to the personal and private details of the patient, the patients have no control on what happens to their data or what those parties are doing with the data in their possession.

Furthermore, these prior systems are usually encoded by the staff of the medical service provider but do not allow the patient to double check or audit data being added to their files. Mistakes in encoding medical data can have dire consequences. These errors can vary from numerical typographical errors to mistakenly encoding medical data to the wrong patient who may or may not have the same name as the original patient.

### SUMMARY OF THE INVENTION

An aspect of the present invention is directed to a system being characterized by one or more central computing devices in a computing environment. The system is more particularly implemented for managing electronic medical records utilizing a set of computer-executable software application services in an electronic computing environment, wherein said system is adapted for keeping, sharing, enumeration, and development of the electronic medical records. The system as claimed comprises a processor and a tangible, non-transitory memory configured to communicate with the processor, wherein the tangible, non-transitory memory has instructions stored thereon that, in response to execution by the processor, cause the processor to perform various computer-implementable operations.

The first operation of the system aspect of the invention is characterized by defining a first permission level associated with a first operation instruction when the first operation instruction is received from at least one patient computing device over a data communication network. The central computing devices and the patient computing device are in operative communication with one another through and/or over the data communication network which may be the Internet.

The second operation of the system aspect of the invention is characterized by obtaining from the patient computing device through the data communication network a second permission level associated with a second operation instruction when the second operation instruction is received from one or more medical data generation sources over the data communication network through which the central computing devices and each medical data generation source one of the medical data generation sources are in operative communication with one another.

The third operation of the system aspect of the invention is characterized by obtaining from the patient communication device through the data communication network verification information in accordance with the second permission level.

The fourth operation of the system aspect of the invention is characterized by determining whether the second operation instruction is to be executed based on the obtained verification information.

In this system aspect of the invention, consistent with one or more implementations thereof as may be disclosed herein, each one of the first and second permission levels is defined based on one or more privacy preference data and one or more access preference data from the patient computing device, and wherein the privacy and access preference data are indicative, respectively, of the privacy and access preferences of a patient associated with the patient computing device.

In this system aspect of the invention, consistent with one or more implementations thereof as may be disclosed herein, the patient associated with the patient computing device is assigned a unique code so as to hide the identity of the patient, and the unique code is replaceable.

Another aspect of the invention is directed to a method for the managing electronic medical records in the computing environment. The method is implementable in a permission-based system for managing electronic medical records utilizing a set of computer-executable software application services in an electronic computing environment, wherein the system is adapted for keeping, sharing, enumeration, and development of the electronic medical records. Preferably, the method is executed by one or more central computing devices in the computing environment, and comprises various computer-executable steps.

In the method aspect of the invention, the first step is characterized by defining, by the central computing devices, a first permission level associated with a first operation instruction when the first operation instruction is received from at least one patient computing device over a data communication network through which the central computing devices and the patient computing device are in operative communication with one another.

In the method aspect of the invention, the second step is characterized by obtaining, by the central computing devices from the patient computing device through the data communication network, a second permission level associated with a second operation instruction when the second operation instruction is received from one or more medical data generation sources over the data communication network through which the central computing devices and each medical data generation source one of the medical data generation sources are in operative communication with one another.

In the method aspect of the invention, the third step is characterized by obtaining, by the central computing devices from the patient communication device through the data communication network, verification information in accordance with the second permission level.

In the method aspect of the invention, the fourth step is characterized by determining, by the central computing devices, whether the second operation instruction is to be executed based on the obtained verification information,

In the method aspect of the invention, consistent with other aspects thereof, each one of the first and second permission levels is defined based on one or more privacy preference data and one or more access preference data from the patient computing device, and wherein the privacy and access preference data are indicative, respectively, of the privacy and access preferences of a patient associated with the patient computing device.

Put differently, one or more implementations of the invention as may be disclosed herein are directed to, and/or involve, a platform, in an electronic computing environment, where patients maintain possession and total authority of their medical records while maintaining the benefits of other systems and methods where data normally isn't in the patient's possession by providing customized access and actions to different MDGS and/or parties requiring the medical data for the benefit of the patient which allows unique synergetic development and enumeration of the data between different sources and parties to increase the effectiveness and impact of the medical records while maintaining patient anonymity and privacy preferences.

In more detail, the platform of the invention allows not just the patient to add medical data, each of which has its own privacy and access preferences, but from other sources as well which goes through an approval process where the patient verifies the data and sets their privacy preferences for that specific data before being added into their medical records. Each medical data generation source or party would have different actions and features available to them through the platform as well as specialized access depending on the patient's preference settings on each medical data. This novel approach of focusing on the capability and availability of each medical data to different parties enables development of the medical data by providing parties who did not originally generate the medical data a unique set of actions like attaching more data such as but not limited to events, notes, and/or warnings to the initial medical data. This feature allows different parties to work together more closely to provide the best outcome for the patient. Furthermore, by segregating medical data generation sources or parties from the custody of the medical records of the patients, the platform operates in stages where medical data or service operation can allow event and status monitoring, and enumeration at the generation stage of data for institutions or governments before the said data are ascribed to a patient to maintain the patient's privacy and anonymity.

It is an object of this invention to provide a cloud-based specialized platform for different MDGS types with the foundation that possession, handling, and responsibility are on the patient for the medical data generated. This allows significant reduction and dependency on information technology infrastructure for MDGSs, especially smaller MDGSs, while maintaining all the required functions for their service as well as important reports and summaries. All MDGS accounts preferably and/or desirably pass through a verification process to ensure the integrity of the medical members in the platform community or computing environment ecosystem. For medical data generated for non-member patients, their records are handled by the platform with pre-set privacy and access settings until the records are claimed by the patient. This means that MDGSs do not maintain a database for any medical records for any patient but only maintains access to those records at the discretion of the patient.

It is an object of this invention to provide a cloud-based DOP medical records system where the patient can assign each data in their medical records a privacy setting to be able to give an openly enhanced personalized presentation to different MDGSs or parties who have varied levels of access on the platform depending on MDGS or party type and relieves the MDGSs of the possessorship and responsibility of the data. Access to the patient's medical records is not primarily limited by who but is limited by what data within the medical records the patient allows to be shown, shared, and viewed based on his settings.

It is yet another object of this invention to allow the medical records of the member patient to be shown to any MDGS or party who has the patient's unique but replaceable code, and the patient's first name. As a security feature, the last name of the member patient or address is not kept in the database. To ensure that the accessed records are indeed of the patient, a visual inspection of the patient and patient image in file is done for verification. If the patient deems that his code is compromised, they may change the code and will be given the option to retain the access given to MDGSs, limit which MDGSs retain their access, or clear all currently authorized MDGSs. The patient may choose to only allow verified MDGSs to gain access to their medical records and not unverified parties of the public.

It is also an object of this invention to allow MDGS to suggest changes into the patient's medical records that goes through an approval process with the patient for verification, setting access and privacy settings for the incoming set of data before applying the changes to their medical records. This access is given by a combination of a code and the patient's first name, followed by a visual inspection of the patient and patient image in file verification. The suggested changes are but not limited to add, edit, note, and/or flag data, into the patient's medical records. So long as the change suggestion has not been approved by the patient, the MDGSs may edit the suggestions that they have created. This method of updating or encoding medical data into medical records prevents the case of hackers using the access of the MDGS to alter or in any way change the medical records of the patient with detrimental effect. The patient can simply disapprove or delete the change suggestion that they deem incorrect or untrue. In some implementations, an MDGS whose account has an abnormally high change suggestion disapproval rate is suspected to misuse and security protocols are engaged to investigate and ensure that the MDGS account is secure.

It is another object of this invention to provide a platform where MDGSs can partner with other MDGSs to be able to forward access to a patient's medical records to another MDGS for a more seamless and secure flow of data in the medical service environment, including computing devices. Any MDGS can also make a draft proposal on a patient that is editable by multiple MDGSs, forwardable to other partner MDGSs, saveable with its own change log, and has not been submitted to the patient.

It is another object of this invention to provide a system and related aspects and/or implementations that allow multiple medical professionals to compile notes on and/or flag each medical data in a medical record to make their opinion, concerns, or warning known to other healthcare professionals who have been given access to the medical data by the patient.

For a better understanding of the invention and to show how the same may be performed in various ways, preferred embodiments and/or implementations thereof will now be described in great detail, by way of non-limiting examples only, with reference to the accompanying drawings.

### BRIEF DESCRIPTION OF DRAWINGS

FIG. 1 is a block diagram showing an example general architecture of the aspects of the invention as claimed.

FIG. 2 is a block diagram showing example detailed architecture of the aspects of the invention as claimed.

FIG. 3 is a flow diagram showing example interactions among the components of the aspects of the invention as claimed, with user privacy and access preferences set to public.

FIG. 4 is a flow diagram showing example interactions among the components of the aspects of the invention as claimed, with user privacy and access preferences set to private.

FIG. 5 is a flow diagram showing an example data flow suitable use in one or more aspects and preferred implementations of the invention as claimed.

FIG. 6 is a block diagram showing an example hardware architecture suitable for use in one or more aspects and preferred implementations of the invention as claimed.

### DETAILED DESCRIPTION OF THE PREFERRED IMPLEMENTATIONS

All the ensuing disclosures and illustrations of the preferred implementations and/or embodiments of one or more aspects of the present invention, along with one or more components, features, elements, or structures thereof, are merely representative for the purpose of sufficiently describing the manner by which the present invention may be carried out into practice in various ways other than the ones outlined in the ensuing enabling description of the present disclosure.

It is to be understood and appreciated, however, that the exemplary implementations used to describe how to make and use one or more aspects of the present invention may be embodied in many alternative forms and should not be construed as limiting the scope of the appended claims in any manner, absent express recitation of those features in the appended claims. All the exemplary drawings, diagrams, tables, formula, and illustrations accompanying the ensuing description should also not be construed as limiting the scope of the appended claims, as accompanied by this description, in any manner.

Unless the context clearly and explicitly indicates otherwise, it is also to be understood that like reference numerals or indicia refer to like elements throughout the ensuing description of the figures and/or drawings of the present disclosure, that the linking term “and/or” includes any and all combinations of one or more of the associated listed items, that the singular terms “a”, “an” and “the” are intended to also include the plural forms, where appropriate, and that some varying terminologies of the same meaning and objective may be interchangeably used throughout the ensuing enabling disclosure of the present invention.

As may be used herein, unless otherwise specified, and consistent with one or more aspects and/or one or more implementations of the present invention, the term “include”, “consist,” or “have” may refer to the corresponding component that can be inherent to exclude other components. All terms, including technical or scientific terms, have the same meaning as generally understood by a person skilled in the art to which the present invention belongs, unless otherwise defined. Commonly used terms, such as herein predefined terms, should be interpreted as being consistent with the meaning of the context of the related art, and are not to be interpreted as ideal or excessively formal meanings unless explicitly defined in the present disclosure of the present invention.

As may be used herein, unless otherwise specified, and consistent with one or more aspects and/or one or more implementations of the present invention, the term “parameter” may refer to a measurable factor that forms part of a set that defines a system and/or sets conditions for operation of the same system. A parameter may be a numerical value, an alphanumerical value, a logical character, a graphical information, or any suitable range type that requires a unit of measure, although various examples are not so limited to such. Each of the plurality of parameters may be based on a corresponding characteristic of the plurality of characteristics. In some preferred examples, the plurality of characteristics may be the plurality of characteristics extracted from any one or more of the herein disclosed steps, processes, operations, tasks, and fields.

As may be used herein, unless otherwise specified, and consistent with one or more aspects and/or one or more implementations of the present invention, the terms “connected to,” “connecting,” “communicating,” “in communication with,” “in operative communication with,” “interconnected,” or “interconnecting” may include direct connection/communication, indirect connection/communication and/or inferred connection/communication between devices/apparatuses/computers. The direct connection/communication may be provided through one or more hardware, software, firmware, electronic and/or electrical links between devices/apparatuses/systems. The indirect connection/communication may be provided through an intervening member such as a component, an element, a circuit, a module, a device, a node device, and an apparatus between or among devices/apparatuses/systems. The inferred connection/communication may be characterized by one device/apparatus/system being connected to or in operative communication with another device/apparatus/system by inference and may include direct and indirect links/connections/communications.

As may be used herein, unless otherwise specified, and consistent with one or more aspects and/or one or more implementations of the present invention, the term “database” may refer to or be characterized by MySQL (an open-source relational database management system), MongoDB (an open-source NoSQL database), PostgreSQL (an object-relational database management system (ORDBMS), or Redis (an in-memory database). Any standard XML (Extensible Markup Language) query language used by most XML databases may also constitute the database system in accordance with various aspects and implementations of the present invention.

As may be used herein, unless otherwise specified, and consistent with one or more aspects and/or one or more implementations of the present invention, the terms “software application” or “application” or “app” may refer to an emulated application or applet, to a web-based application, or to a mobile-based application, and may specifically refer to an executable computer software program or software application program that enables services and content associated with one or more implementations of the herein disclosed method, system, computer program product, intermediate system, further intermediate system comprising multiple intermediate systems, data stream, and data structure. The application may be a mobile app or any other application that is executable via any one or more of client computing devices via a server or via a network of computers. The computer-executable program or programs which may constitute the application may be a self-contained software or is a component of or contained by another program or programs, any of which may be implemented by one or more hardware, software, firmware and/or cloud computing or cloud related computing resources comprising one or more infrastructure stacks and one or more infrastructure components such as application servers, file servers, directory servers, web servers, network servers, database servers, and the like.

As may be used herein, unless otherwise specified, and consistent with one or more aspects and/or one or more implementations of the present invention, the terms “patient computing device” may refer to a wireless mobile or non-mobile data communication device such as a mobile phone, a smart phone, a personal digital assistant (PDA) device, a tablet device, a phablet device, a desktop computer, a laptop computer, and the like. The patient computing device may be connected to the herein disclosed data communications network and may access the herein disclosed platform. The patient computing device may be used by a client to gain access to the herein disclosed digital services, accessible via a network, of the present invention.

As may be used herein, unless otherwise specified, and consistent with one or more aspects and/or one or more implementations of the present invention, the term “processor” may refer to logic circuitry and/or processing core that is configured to implement computer programming instructions that cause an electronic device to perform various functions. Unless specifically stated otherwise, the term “processor” can refer to a single processing device or to multiple processing devices that together implement various steps of a process.

As may be used herein, unless otherwise specified, and consistent with one or more aspects and/or one or more implementations of the present invention, the terms “article of manufacture” or “computer-readable medium” may refer to any medium that participates in providing data (e.g., instructions, data structures) which may be read by a computer, a processor or a like device. Such a medium may take many forms, including but not limited to, non-volatile media, volatile media, and transmission media. Non-volatile media include, for example, optical or magnetic disks and other persistent memory. Volatile media include dynamic random-access memory (DRAM), which typically constitutes the main memory. Transmission media include coaxial cables, copper wire and fiber optics, including the wires that comprise a system bus coupled to the processor. Transmission media may include or convey acoustic waves, light waves and electromagnetic emissions, such as those generated during radio frequency (RF) and infrared (IR) data communications. Common forms of computer-readable media include, for example, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH-EEPROM, any other memory chip or cartridge, a carrier wave as described hereinafter, or any other medium from which a computing device or computing system can read.

Likewise, unless otherwise specified, and consistent with one or more aspects and/or one or more implementations of the present invention, the terms “memory” or “memory system” should be interpreted broadly to encompass any electronic component capable of storing electronic information. The term memory may refer to various types of processor-readable media such as random-access memory (RAM), read-only memory (ROM), non-volatile random-access memory (NVRAM), programmable read-only memory (PROM), erasable programmable read-only memory (EPROM), electrically erasable PROM (EEPROM), flash memory, magnetic or optical data storage, registers, etc. Memory is said to be in electronic communication with a processor if the processor can read information from and/or write information to the memory. Memory that is integral to a processor is in electronic communication with the processor.

As may be used herein, unless otherwise specified, and consistent with one or more aspects and/or one or more implementations of the present invention, the terms “instructions,” “operation instructions”, “codes,” and “program codes” should further be interpreted broadly to include any type of computer-readable statement(s). For example, the terms “instructions” and “codes” may refer to one or more programs, routines, sub-routines, functions, procedures, etc. “Instructions” and “codes” may comprise a single computer-readable statement or multiple computer-readable statements.

As may be used herein, unless otherwise specified, and consistent with one or more aspects and/or one or more implementations of the present invention, the term “module” may refer to any hardware, software, firmware, electronic control component, processing logic, and/or processor device, individually or in any combination, including without limitation: application specific integrated circuit, an electronic circuit, a processor (shared, dedicated, or group depending in privacy settings) and memory that executes one or more software or firmware programs, a combinational logic circuit, and/or other suitable and re-configurable components or systems of components that provide the herein disclosed and/or described functionalities.

It is to be understood and appreciated by a skilled person or a person having ordinary skills in the art to which the present invention belongs that the herein described modules are merely presented in segregated format based on their intended functions for the sake of illustrating how they are relevant to the implementations and/or aspects of the present invention. The herein described modules are merely illustrative and can be fewer or greater in number, as it is well known in the art of computing that such program codes representing various functions of different operably connected modules can be combined or segregated in any suitable but efficient manner.

It is also to be understood and appreciated that, in any portion or portions of the herein disclosure, the use of ordinal terms such as “first,” “second,” “third,” and so forth, is used herein to distinguish elements, features, components, calculations or steps from one another and should not also be construed as limiting the scope of the appended claims, and that these and such other ordinal terms that may appear in the ensuing description of the one or more aspects of the present invention are not indicative of any particular order of elements, features, calculations, components or steps to which they are attached. For example, a first element could be termed a second element or a third element. Similarly, a second element could be termed a first element or a third element. All these do not depart from the scope of the herein disclosure and its accompanying claims.

As may be used herein, unless otherwise specified, and consistent with one or more aspects and/or one or more implementations of the present invention, the term “system” may refer to the hardware and software environment in which application programs operate. The system is thus defined by the combined effect of native operating hardware and software and any other operating environment. The operating environment or the computing environment may be a software layer existing between any native operating hardware and any framework. Still, such a layer may not exist or may be different.

As may be used herein, and consistent with one or more implementations of the present invention, the terms “computer-implemented method” may refer to the method that may be carried out in a computer or computing system, e.g., a computer program executed by a CPU. A computer, as used herein, refers to a computing device that can be programmed to perform a set of arithmetic or logical operations automatically. Computers or computing systems, as used herein, may include personal computers, workstations, servers, mainframes and supercomputers. The computers or computing systems can be a stand-alone system, networked system or a virtual machine residing in a computing network and/or a mobile network.

As may be used herein, and consistent with one or more implementations of the present invention, the term “processor” may refer to a central processing unit or “CPU” which is a computing device that can read a computer-executable program from a computer memory (e.g., ROM or other computer memory) and perform a set of steps according to the computer-executable programs associated with computing devices, or servers, or server computers. The terms “computer readable medium” in totality may refer to any device or system for storing and providing information to a computer processor, examples of which may include drives and servers, by way of examples, for exchanging packets over mobile communication networks.

As may be used herein, and consistent with one or more implementations of the present invention, the term “node” may refer to any equipment that is adapted, configured, arranged, and/or operably designed to communicate directly, or even indirectly in some scenarios, with one or more wireless communication devices and/or with other network side nodes or equipment in the wireless communication network to enable and/or provide wireless access to the wireless communication device and/or to perform various functions in the wireless communication network through the use of processing circuitries.

It is be to be understood and appreciated that one or more aspects of the present invention may be implemented using hybrid on-demand protocols that manage all the exhaustive pulling, pushing and synchronization of all medical data or information or patient records, in either structured or unstructured format, contained into one-single source platform, cleaned, synchronized, encrypted using hybrid networks and corrected for duplicate, and then made available on-demand, electronically, and based on permission settings to authorized healthcare providers, professionals, partners and patients alike, with anytime, anyplace access via hybrid mixed of offline client-server or software application executing on a computing device such as a laptop computer that is connected to a network, or mobile application executing on a mobile device, or via cloud private/public computing as SaaS (Software-as-Service) or any IP (internet protocol) or telecommunication internetwork protocol such as Wi-Fi or IEEE 802.11 network of standards, Wimax or IEEE 802.16 family of wireless-networks standards, GPRS, 3G/4G/Long Term Evolution/5G standards, or any future generations and standards of data communication networks.

Accordingly, and in some implementations, aspects of the present invention may utilize electronic data warehouse containing most complete and detailed lifelong medical historical information, including patient records or medication usage of a hospital, a medical clinic, a laboratory, or any medical data generation source (MDGS) for that matter and for purposes that may be disclosed or undisclosed in the ensuing description, made available on-demand and electronically to authorized healthcare providers, professionals, partners and patients alike, with anytime, anyplace access via web or internet protocol (IP) or telecommunication internetwork standard protocol.

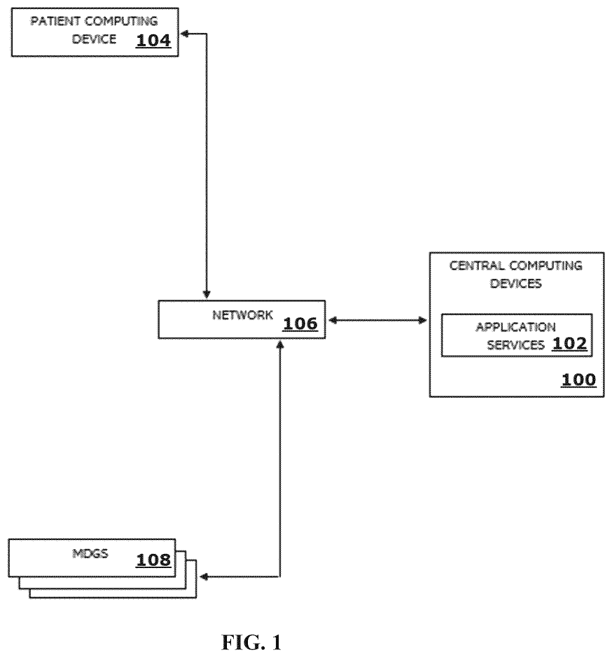

One aspect of the present invention is directed to a method being executed by one or more central computing devices 100 in a computing environment. This is illustrated in the block diagram of FIG. 1 that illustrates an example general architecture of the aspects of the present invention as claimed. Accordingly, the method, which comprises various operations or steps, may be particularly implemented in a permission-based system for managing electronic medical records utilizing a set of computer-executable software application services 102 in the computing environment or electronic computing environment. The application services 102 may be maintained by the central computing devices 100 in the form of electronic platform. Consistent with one or more aspects and implementations of the present invention, the system is adapted for keeping, sharing, enumeration, and development of the electronic medical records.

The herein disclosed platform 102, maintained by the central computing devices 100, is where the patient may be able to maintain possession and total authority of his medical records that may be developed over time. All these while maintaining the benefits of other systems and methods where data normally isn't in the patient's possession by providing customized access and actions to the different medical data generation sources 108 and/or parties requiring the medical data for the benefit of the patient which allows unique synergetic development and enumeration of the data between different sources 108 and parties to increase the effectiveness and impact of the medical records while maintaining patient anonymity and privacy preferences, including access preferences. The sources 108 may refer to, collectively in most cases, source computing devices and human professionals such as doctors and medical technologists who are operating the source computing devices.

The first step of the method aspect of the invention is characterized by, defining, by the central computing devices 100, a first permission level associated with a first operation instruction when the first operation instruction is received from at least one patient computing device 104 over a data communication network 106. An example of the first operation instruction is an instruction to upload personal medical data of a patient using for example the patient computing device 104 voluntarily or at his discretion, without reference to any external data sources. The central computing devices 100 and the patient computing device 104 are in operative communication with one another through and/or over the data communication network 106 which may be the Internet.

The second step of the method aspect of the invention is characterized by, obtaining, by the central computing devices 100 from the patient computing device 104 through the data communication network 106, a second permission level associated with a second operation instruction when the second operation instruction is received from one or more medical data generation sources 108 over the data communication network 106, which may be the Internet, through which the central computing devices 100 and each medical data generation source 108 one of the medical data generation sources 108 are in operative communication with one another. An example of the second operation instruction is an instruction to encode data indicative of a prescription from a medical data generation source 108 which may be, by way of example, a network connected physician's computing device or hospital's computer terminal.

Consistent with one or more aspects and herein disclosed implementations of the present invention, the permission level of any one or more of the first and second permission levels may be at least one of the following configurable permissions based on individual user preferences: a read only permission, a write only permission, a read and write permission, an erase permission, a no permission, a flag permission, an access permission, a sharing permission, a recording permission, an admin permission, an execute permission, an uploading permission, a downloading permission, and role creation permission. In an implementation, the private manipulation of electronic medical data or records by the patient operating the patient computing device 104 based on one or more of permissions may be managed using a synchronization module executing on the central computing device 100. The synchronization module may be adapted for processing a set of synchronization requests from different data manipulation operation instructions. In accordance with a set of rules, which may pre-defined, the set of synchronization requests being queued for execution against each and every new medical data or record sought to be recorded into the platform 102 which may be, or may consist of, software application services 102.

The third step of the method aspect of the invention is characterized by, obtaining, by the central computing devices 100 from the patient communication device 104 through the data communication network 106, verification information in accordance with the second permission level arising from the second operation instruction caused to be generated and transmitted by the medical data generation sources 108.

The fourth step of the method aspect of the invention is characterized by, determining, by the central computing devices 100, whether the second operation instruction is to be executed based on the obtained verification information. The verification information may contain the authorization of the patient for his particular medical record to be added into the herein disclosed platform maintained by the central computing devices 100.

In the method aspect of the present invention, each one of the first and second permission levels is defined based on one or more privacy preference data and one or more access preference data from the patient computing device 104, and wherein the privacy and access preference data are indicative, respectively, of the privacy and access preferences of a patient associated with the patient computing device 104. It should be noted that the patient computing device 104 does not store the medical data or records of the patient operating it; nor does the MDGS 108 providing some of the same data and records. Instead, the electronic medical records or data are stored in the central computing devices 100, and these records or data are accessible by any computing devices through the platform 102, over the data communication network 106 which may be the Internet. It is the patient who owns his medical records or data, and has the total authority to control, access, and manipulated the same.

In the method aspect of the present invention, consistent with one or more implementations thereof, the aforementioned steps may further include performing an operation corresponding to the executing second operation instruction if the second operation instruction is determined to be executed.

In the method aspect of the present invention, as disclosed the patient associated with the patient computing device 104 is assigned a unique and replaceable code so as to hide the identity of the patient. The code may be a numeric code (or a serial number), an alphanumeric code, a bar code, a quick response (QR) code, an image, an alias, or any of such forms that can be derived from third party computing systems.

The herein disclosed provisions of one or more aspects of the present invention, and their respective one or more implementations, ensure that, apart from voluntarily adding medical data into the system having their respective privacy and access preferences, the patient or user may also manage medical data from other sources that can be subjected to approval process where the patient verifies the data and sets his privacy and/or access preferences for that specific data before the same data can be added into his developing medical records or data.

Consistent with one or more aspects and implementations of the present invention, each medical data generation source or party 108 may have different actions and features available to them through the platform 102 as well as specialized access depending on the patient's preference settings on each medical data, on each data type, or on each purpose-specific data. This approach of focusing on the capability and availability of each medical data to different sources or parties 108 enables development of the medical data by providing parties who did not originally generate the medical data a unique set of actions like attaching more data such as but not limited to events, notes, and/or warnings to the initial medical data. In this regard, different parties, mainly including the sources 108, are allowed to work together more closely to provide the best outcome for the patient. Furthermore, by segregating medical data generation sources or parties from the custody of the medical records of the patients, the platform 102 operates in stages where medical data or service operation can allow event and status monitoring, and enumeration at the generation stage of data for institutions or governments before the said data are ascribed to the concerned patient to maintain the patient's privacy and anonymity.

The application services 102 residing on the central computing devices 100, or network connected central computing devices 100, are preferably arranged to have (1) a framework for collecting electronic medical data based on operation instructions; (2) a framework for providing the electronic medical data based on demand; (3) a framework for providing a graphical representation of the electronic medical data; and (4) a framework for providing a graphical representation of the modules corresponding to the set of software application services based on a healthcare-specific workflow model. The term “based on demand” may be characterized by the process through which users, sources, or parties may selectively choose the set of the software application services that they need. The application services 102 may refer to the herein disclosed platform, and hence the terms application services and platform 102 may be interchangeably used throughout the ensuing disclosure.

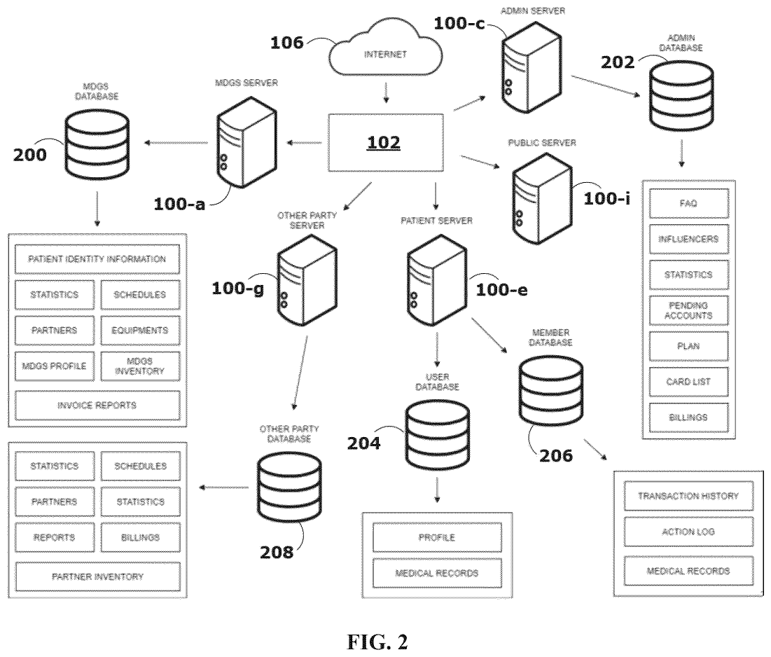

Referring to FIG. 2, consistent with one or more aspects and implementations of the present invention, there is shown block diagram showing example detailed architecture of the aspects of the present invention, and particularly illustrating the cloud-based specialized platform 102 for different MDGS types with the foundation that possession, handling, and responsibility is on the patient for the electronic medical data generated. This allows significant reduction and dependency on information technology infrastructure for MDGSs 108, especially smaller MDGSs 108, while maintaining all the required functions for their service as well as important reports, summaries, and the like.

In FIG. 2, consistent with one or more aspects and implementations of the present invention, there is illustrated a preferred arrangement of components of the present invention, mainly including the server computing devices or servers 100-a, 100-c, 100-e, 100-g, 100-i that are in operative communication with one another through the data communication network 106 which may be the Internet. The MDGS server 100-a may be configured to operably maintain an MDGS database 200. The MGDS database 200 operably functioning as a data store for the MDGS server 100-a connected to the platform 102 preferably stores electronic data associated with one or more of, or any suitable combination of, the following: patient identity information, statistics, schedules, partners, equipment, MDGS profile, MDGS inventory, and invoice reports.

The admin server 100-c may be configured to operably maintain an admin database 202. The admin database 202 operably functioning as a data store for the admin server 100-c connected to the platform 102 preferably stores data associated with one or more of, or any suitable combination of, the following: FAQs, influencers, statistics, pending accounts, plan, card list and billings, and as well as payments, ledgers, and the like.

The patient server 100-e may be configured to operably maintain a user database 204 and a member database 206. The user database 204 operably functions as a data store for the patient server 100-e connected to the platform 102 preferably stores data associated with one or more of, or any suitable combination of, the user profile and user medical records. The member database 206 operably functioning as a data store for the patient server 100-e connected to the platform 102 preferably stores data associated with one or more of, or any suitable combination of, the following: transaction history, action log, and medical records, as well as predicted data from predictive models, neural networks, multi-metric indices, and the like.

The other party server 100-g may be configured to operably maintain a third party or other party database 208. The other party database 208 operably functioning as a data store for the other party server 100-g connected to the platform 102 preferably stores data associated with one or more of, or any suitable combination of, the following: statistics, partners, reports, schedules, billings, and partner inventory. The example architecture in FIG. 2 also shows a public server 100-i that is open to the public for accessing general medical information that may be stored in the platform 102 of the present invention.

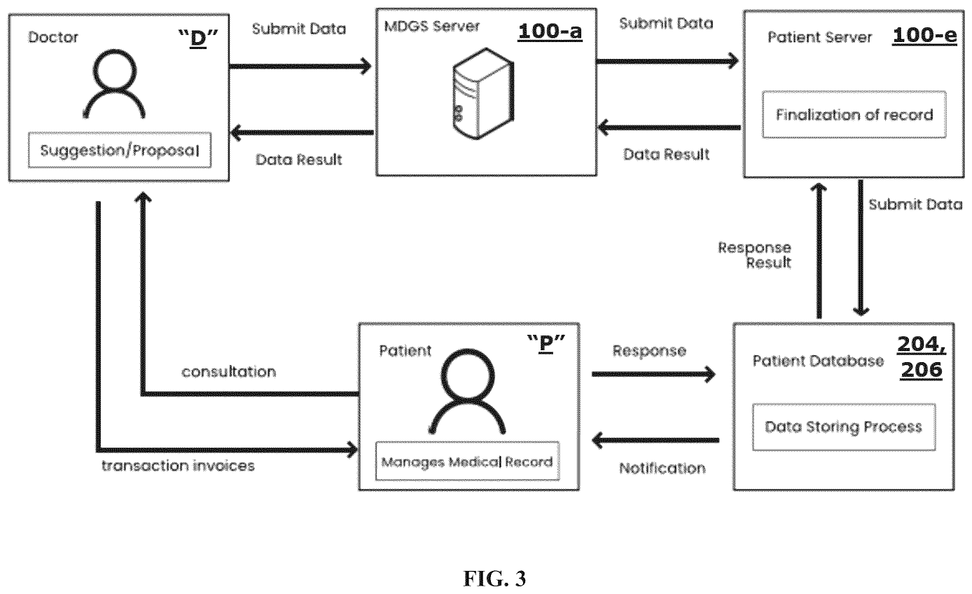

Referring now to FIG. 3, there is shown a flow diagram illustrating example interactions among the components of the aspects of the invention as claimed, with user privacy and access preferences set to public. The interaction may commence when the patient “P,” who manages his own medical records, enters into consultation with the doctor “D” using, via, or through the platform 102 of the present invention. Any such data that may be voluntarily and personally created and managed by the patient “P” may constitute the first permission level of one or more aspects and implementations of the present invention. Consistent with one or more aspects and implementations of the present invention, one or more software application services 102 in the set of software application services 102 are caused by the central computing devices 100 to render on the patient computing device 104 and the medical data generation sources 108, particularly on the MDGS computer terminals or workstations 108, graphical representations of at least the medical records, the privacy preference data, and the access preference data based on healthcare-specific workflow models. In some implementations, the healthcare-specific workflow models are based on a plurality of computer-executable healthcare modules that may be utilized in the herein disclosed aspects and implementations of the present invention as may be found in the present disclosure.

The consultation initiated by the patient “P” may constitute the first operation instruction of the present invention. In some implementations, one or more of the permissions arising from or associated with the first and second permission levels have validity periods or expiration. This configuration gives more flexibility for the users of, or parties to, the platform community in handling electronic medical records, especially in cases where a series of diagnoses, treatments, and/or simple consultations for a certain period is required under certain circumstances associated with the medical condition of the patient “P” operating the device 104.

In turn, the doctor “D” can have his diagnosis based on the consultation initiated by the patient “P.” The diagnosis of the doctor “D,” for example, may form part of the medical data that can be submitted by the doctor “D” to the MDGS server 100-a. This submission initiated by the doctor “D,” who may be operating one or more of the MDGSs 108, may constitute the second operation instruction of the present invention. This event may trigger the patient server 100-e to store the medical data from the MDGS server 100-a into the patient database 204, 206. The stored medical may in turn be communicated with the patient “P” through the network connected patient computing device 104.

The data storing process or routine that can be performed by way of example by the patient server 100-e preferably requires verification process. To do this, the patient “P” may first receive a notification about the new medical record being sought to be added from the MGDS server 100-a and originating from the end of the doctor “D.” The patient “P” at this point may send his response, i.e., whether to allow or permit the storing of his medical data, into the patient database 204, 206. In one implementation, without necessarily limiting the scope of the present invention as claimed, the response result may be communicated with the computing device of the doctor “D.”

The response result in this case may pass through the patient server 100-e and the MDGS server 100-a for processing. Consistent with one or more aspects and implementations of the present invention, the notification communicated with the patient “P” and the response result obtain from the patient “P” may form part of the verification information of the present invention. One or more of the parameters associated with the verification information and the verification process that carries them may be pre-configured or reconfigurable based on the desired level of verification and/or authentication by the patient “P.” A multi-verification process may be employed for greater security of personal data owned and managed by the patient “P.”

As one example final interaction between the doctor “D” and the patient “P” through their respective network connected communication and/or computing devices, a transaction invoice may be caused to be generated by the doctor “D” and which in turn may be communicated with patient “P.” Various forms of settlements or payment transactions may be integrated into the platform 102 of the present invention, and these may include by way of examples, debit card payment transactions, credit card payment transactions, fund transfer transactions, and the like.

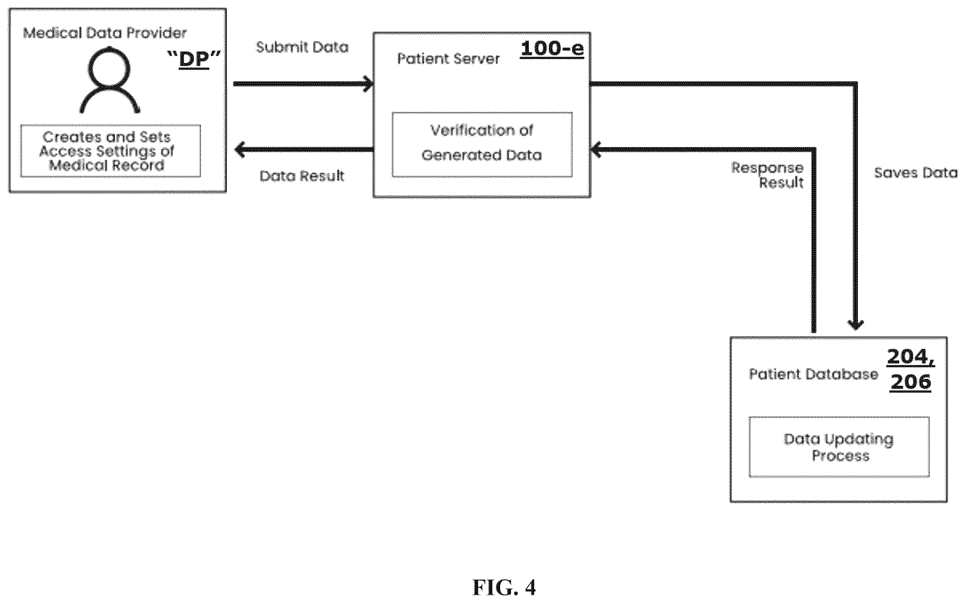

In FIG. 4, there is shown a flow diagram illustrating example interactions among the components of the aspects of the present invention as claimed, with user privacy and access preferences set to private. In some implementations, the medical data provider “DP,” which may be operating and/or maintaining the MDGS 108 of the present invention as claimed and disclosed, may create and/or set the access settings of the medical record. In this case, the medical data may be shared by and between the MDGS 108 and the patient server 100-e. This event may trigger the patient server 100-e to perform verification of the generated medical data and data updating and storing process against the patient database 204, 206 for each and every successful verification and/or authentication processing.

The medical data result arising from these processes, along with other processes as the case may be, may then be communicated over the data communication network 106 by and between the patient server 100-e and the MDGS 108 operated by the medical data provider “DP.” Accordingly, the software application services 102 may include a service for allowing the medical data generation sources 108 to upload and/or cause to be stored the medical data in the central computing devices 102. The medical data may be characterized by different formats. In which case, it is desirable that the software application services 102 be configured to include a service for converting the formats into one or more classified formats for private viewing.

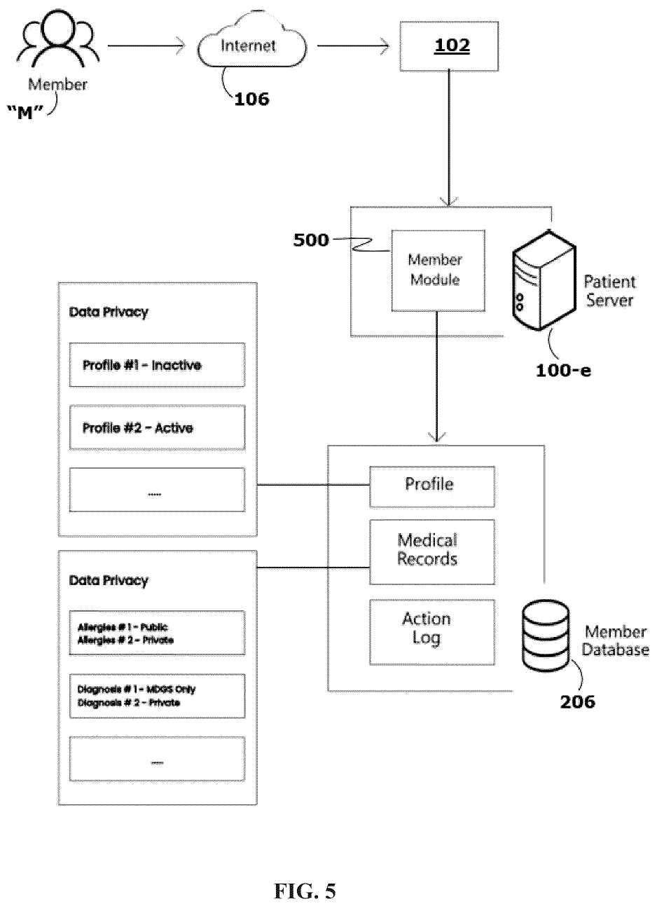

Referring now to FIG. 5, there is shown a flow diagram that illustrates an example data flow suitable use in one or more aspects and preferred implementations of the present invention as claimed. As exemplary illustrated, any member “M” of the platform community may access the platform 102 through the data communication network 106. The member module 500 being operably maintained by the patient server 100-e may be configured suitable for use in managing the profile of the member “M,” the medical records associated with and/or owned by the member “M,” and the action logs, any one or more or all of which may be stored in the member database 206. The privacy and/or access settings applied to each medical data or medical record of the member “M” may be set by the member “M.”

For example, the privacy of two data associated with two allergies may be set independently of one another, i.e., the first allergy may be set to public while the second allergy may be set to private. The same principle and logic may be applied to the first and second diagnosis as shown, wherein one of the diagnoses may be accessed by the MDGS 108 of the present invention only while the other may be set to private mode of data operation. Consistent with these provisions of the disclosed aspects and implementations of the present invention, the privacy preference data and one or more access preference data vary for each medical data generation sources 108 of the medical data generation sources 108. In some implementations, wherein the privacy preference data and one or more access preference data may be configurable on-demand. In some implementations, consistent with one or more aspects of the present invention as claimed and disclosed, any one or more of pluralities of the privacy preference data and access preference data are pre-configured in the central computing devices 100 executing the platform 102 and residing in the computing environment.

In a permission-based system for managing electronic medical records utilizing a set of computer-executable software application services in an electronic computing environment, the system being adapted for keeping, sharing, enumeration, and development of the electronic medical records, an article of manufacture is also disclosed, said article of manufacture comprising a processor-readable storage medium having stored therein program codes corresponding to the application services, wherein the program codes are executable by one or more processors to implement a process comprising different computer-executable and implementable steps.

The first step of the article of manufacture aspect of the invention is characterized by defining a first permission level associated with a first operation instruction when the first operation instruction is received from at least one patient computing device over a data communication network. The central computing devices and the patient computing device are in operative communication with one another through and/or over the data communication network which may be the Internet.

The second step of the article of manufacture aspect of the invention is characterized by obtaining from the patient computing device through the data communication network, a second permission level associated with a second operation instruction when the second operation instruction is received from one or more medical data generation sources over the data communication network through which the central computing devices and each medical data generation source one of the medical data generation sources are in operative communication with one another.

The third step of the article of manufacture aspect of the invention is characterized by obtaining from the patient communication device through the data communication network, verification information in accordance with the second permission level.

The fourth step of the article of manufacture aspect of the invention is characterized by determining whether the second operation instruction is to be executed based on the obtained verification information.

In the article of manufacture aspect of the present invention, each one of the first and second permission levels is defined based on one or more privacy preference data and one or more access preference data from the patient computing device, and wherein the privacy and access preference data are indicative, respectively, of the privacy and access preferences of a patient associated with the patient computing device.

In the article of manufacture aspect of the present invention, consistent with one or more implementations thereof, the steps further include performing an operation corresponding to the executing second operation instruction if the second operation instruction is determined to be executed.

In the article of manufacture aspect of the present invention, as disclosed the patient associated with the patient computing device is assigned a unique and replaceable code so as to hide the identity of the patient. The code may be a numeric code (or a serial number), an alphanumeric code, a bar code, a QR code, an image, an alias, or any of such forms that can be derived from any third-party computing systems.

In the article of manufacture aspect of the present invention, consistent with one or more implementations thereof, a permission level of any one or more of the first and second permission levels is selected from at least one of a read only permission, a write only permission, a read and write permission, an erase permission, a no permission, a flag permission, an access permission, a sharing permission, a recording permission, an admin permission, an execute permission, an uploading permission, a downloading permission, and role creation permission.

In the article of manufacture aspect of the present invention, consistent with one or more implementations thereof, one or more of the permissions have validity periods.

In the article of manufacture aspect of the present invention, consistent with one or more implementations thereof, the one or more privacy preference data and one or more access preference data vary for each medical data generation sources of the medical data generation sources.

In the article of manufacture aspect of the present invention, consistent with one or more implementations thereof, the one or more privacy preference data and one or more access preference data are configurable on-demand.

In the article of manufacture aspect of the present invention, consistent with one or more implementations thereof, any one or more of pluralities of the privacy preference data and access preference data are pre-configured in the central computing devices residing in the computing environment.

In the article of manufacture aspect of the present invention, consistent with one or more implementations thereof, the central computing devices have a synchronization module adapted for processing a set of synchronization requests, the set of synchronization requests being queued in accordance with a set of rules.

In the article of manufacture aspect of the present invention, consistent with one or more implementations thereof, one or more software application services in the set of software application services are caused by the central computing devices to render on the patient computing device and the medical data generation sources graphical representations of at least the medical records, the privacy preference data, and the access preference data based on healthcare-specific workflow models.

In the article of manufacture aspect of the present invention, consistent with one or more implementations thereof, the healthcare-specific workflow models are based on a plurality of computer-executable healthcare modules.

In the article of manufacture aspect of the present invention, consistent with one or more implementations thereof, the software application services include a service for allowing the medical data generation sources to upload the medical data in the central computing devices, and wherein the medical data are characterized by different formats.

In the article of manufacture aspect of the present invention, consistent with one or more implementations thereof, the software application services include a service for converting the formats into one or more classified formats.

Yet another aspect of the present invention is directed to a permission based system for managing electronic medical records utilizing a set of computer-executable software application services in an electronic computing environment, the system being adapted for keeping, sharing, enumeration, and development of the electronic medical records, the system comprising a processor and a tangible, non-transitory memory configured to communicate with the processor, the tangible, non-transitory memory having instructions stored thereon that, in response to execution by the processor, cause the processor to perform various computer-implementable operations.

The first operation of the system aspect of the invention is characterized by defining a first permission level associated with a first operation instruction when the first operation instruction is received from at least one patient computing device over a data communication network. The central computing devices and the patient computing device are in operative communication with one another through and/or over the data communication network which may be the Internet.

The second operation of the system aspect of the invention is characterized by obtaining from the patient computing device through the data communication network, a second permission level associated with a second operation instruction when the second operation instruction is received from one or more medical data generation sources over the data communication network through which the central computing devices and each medical data generation source one of the medical data generation sources are in operative communication with one another.

The third operation of the system aspect of the invention is characterized by obtaining from the patient communication device through the data communication network, verification information in accordance with the second permission level.

The fourth operation of the system aspect of the invention is characterized by determining whether the second operation instruction is to be executed based on the obtained verification information.

In the system aspect of the present invention, each one of the first and second permission levels is defined based on one or more privacy preference data and one or more access preference data from the patient computing device, and wherein the privacy and access preference data are indicative, respectively, of the privacy and access preferences of a patient associated with the patient computing device.

In the system aspect of the present invention, as disclosed the patient associated with the patient computing device is assigned a unique and replaceable code so as to hide the identity of the patient. The code may be a numeric code (or a serial number), an alphanumeric code, a bar code, a quick response (QR) code, an image, an alias, or any of such forms that can be derived from third party computing systems.

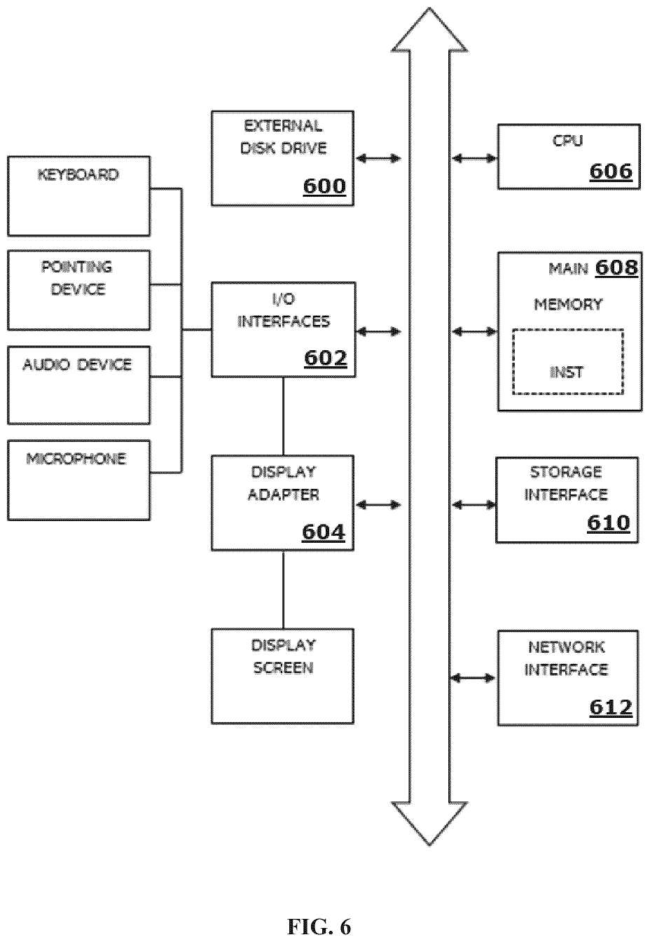

In FIG. 6, there shown a block diagram illustrating an example hardware architecture suitable for use in one or more aspects and preferred implementation of the present invention as claimed. By way of examples, any one of the herein disclosed central computing devices 100 may have the illustrated hardware architecture. As exemplary shown, such hardware may include the external drive 600, the input and output (I/O) interfaces 602 for a keyboard, a pointing device, an audio device, and a microphone, and the display adapter 604 interfaced to a display screen. Likewise included in this example hardware architecture are the CPU 606, the main memory 608 storing instructions, the storage interface 610, and the network interface sss.

Although the preferred embodiments and/or implementations have been described in detail above, it should be understood that various changes, substitutions, and alterations can be made herein without departing from the scope of the present invention. For example, although the present invention has been described and illustrated primarily in relation to data networks, it should be understood that the present invention is in no way limited to such data networks and may alternatively be implemented on other types of networks. It should be understood that the application of the present invention is not limited only to the present disclosure. Rather, the present invention may be suitably implemented to perform all forms of medical and other transactions over a data communications network that will be apparent to those skilled in the art.

Furthermore, it should be noted that the present invention can be implemented using virtually any computer system or other networking system and virtually any available programming language. Thus, the implementations of the present invention are not limited to the computing environment illustrated in this document. Put differently, other examples of changes, substitutions, and alterations are readily ascertainable by one skilled in the art and could be made without departing from the spirit and scope of the present invention as clearly defined by the following claims as appended to this disclosure.

What is claimed is:

1. A permission-based system for managing electronic medical records in a computing environment, the system being adapted for keeping, sharing, enumeration, and development of the electronic medical records, the system comprising a processor and a tangible, non-transitory memory configured to communicate with the processor, the tangible, non-transitory memory having instructions stored thereon that, in response to execution by the processor, cause the processor to perform:
defining a first permission level associated with a first operation instruction when the first operation instruction is received from at least one patient computing device over a data communication network through which the central computing devices and the patient computing device are in operative communication with one another; obtaining from the patient computing device through the data communication network a second permission level associated with a second operation instruction when the second operation instruction is received from one or more medical data generation sources over the data communication network through which the central computing devices and each medical data generation source one of the medical data generation sources are in operative communication with one another;
obtaining from the patient communication device through the data communication network verification information in accordance with the second permission level; and
determining whether the second operation instruction is to be executed based on the obtained verification information,
wherein each one of the first and second permission levels is defined based on one or more privacy preference data and one or more access preference data from the patient computing device, wherein the privacy and access preference data are indicative, respectively, of the privacy and access preferences of a patient associated with the patient computing device, wherein the patient associated with the patient computing device is assigned a unique code so as to hide the identity of the patient, and wherein the unique code is replaceable.

2. The system according to claim 1, the processor is caused to further perform an operation corresponding to the executing second operation instruction if the second operation instruction is determined to be executed.

3. The system according to claim 1, wherein permission level of any one or more of the first and second permission levels is selected from at least one of a read only permission, a write only permission, a read and write permission, an erase permission, a no permission, a flag permission, an access permission, a sharing permission, a recording permission, an admin permission, an execute permission, an uploading permission, a downloading permission, and role creation permission.

4. The system according to claim 3, wherein one or more of the permissions have validity periods.

5. The system according to claim 1, wherein the one or more privacy preference data and one or more access preference data vary for each medical data generation sources of the medical data generation sources.

6. The system according to claim 1, wherein the one or more privacy preference data and one or more access preference data are configurable on-demand.

7. The system according to claim 1, wherein any one or more of pluralities of the privacy preference data and access preference data are pre-configured in the central computing devices residing in the computing environment.

8. The system according to claim 1, wherein the central computing devices have a synchronization module adapted for processing a set of synchronization requests, the set of synchronization requests being queued in accordance with a set of rules.

9. The system according to claim 1, wherein one or more software application services in the set of software application services are caused by the central computing devices to render on the patient computing device and the medical data generation sources graphical representations of at least the medical records, the privacy preference data, and the access preference data based on healthcare-specific workflow models.

10. The system according to claim 1, wherein the healthcare-specific workflow models are based on a plurality of computer-executable healthcare modules.

11. The system according to claim 1, wherein the software application services include a service for allowing the medical data generation sources to upload the medical data in the central computing devices, and wherein the medical data are characterized by different formats.

12. The system according to claim 11, wherein the software application services include a service for converting the formats into one or more classified formats.

13. In a permission-based system for managing electronic medical records utilizing a set of computer-executable software application services in an electronic computing environment, the system being adapted for keeping, sharing, enumeration, and development of the electronic medical records, a method for the managing electronic medical records in the computing environment, the method being executed by one or more central computing devices in the computing environment, the method comprising the steps of:
defining, by the central computing devices, a first permission level associated with a first operation instruction when the first operation instruction is received from at least one patient computing device over a data communication network through which the central computing devices and the patient computing device are in operative communication with one another;
obtaining, by the central computing devices from the patient computing device through the data communication network, a second permission level associated with a second operation instruction when the second operation instruction is received from one or more medical data generation sources over the data communication network through which the central computing devices and each medical data generation source one of the medical data generation sources are in operative communication with one another;
obtaining, by the central computing devices from the patient communication device through the data communication network, verification information in accordance with the second permission level; and
determining, by the central computing devices, whether the second operation instruction is to be executed based on the obtained verification information,
wherein each one of the first and second permission levels is defined based on one or more privacy preference data and one or more access preference data from the patient computing device, and wherein the privacy and access preference data are indicative, respectively, of the privacy and access preferences of a patient associated with the patient computing device.
